(19) United States
(12) Patent Application Publication
(10) Pub. No.: US 2026/0053516 A1
(43) Pub. Date: Feb. 26, 2026

(54) PROTECTIVE SLEEVE FOR USE WITH AN INTRAVASCULAR LITHOTRIPSY DEVICE

(71) Applicant: BOLT MEDICAL, INC., Carlsbad, CA (US)

(72) Inventors: Alan Duong, San Diego, CA (US); Swathi Rangarajan, San Marcos, CA (US); Eric Schultheis, San Clemente, CA (US)

(73) Assignee: BOLT MEDICAL, INC., Carlsbad, CA (US)

(21) Appl. No.: 19/306,365

(22) Filed: Aug. 21, 2025

Related U.S. Application Data
(60) Provisional application No. 63/685,861, filed on Aug. 22, 2024.

(51) Int. Cl. A61B 17/22; A61B 17/00; A61M 25/10

(52) U.S. Cl. CPC A61B 17/22004; A61B 17/00234; A61M 25/10; A61B 2017/00238; A61B 2017/00305; A61B 2017/00557; A61B 2017/22051; A61M 2025/1081; A61M 2210/12

(57) ABSTRACT

A protective sleeve (229) for protecting a balloon (204) of an intravascular lithotripsy catheter system (200) only prior to the balloon (204) being positioned within a body (107) of a patient (109), includes a sleeve body (229B) that is configured to be removably positioned about the balloon (204). The sleeve body (229B) includes a sleeve proximal end (229P) and a sleeve distal end (229D). The sleeve body (229B) includes a tapered region (264C) having a tapered diameter (262V) that decreases in a direction from the sleeve proximal end (229P) toward the sleeve distal end (229D).

### CROSS-REFERENCE TO RELATED APPLICATIONS

This application claims the benefit of priority under 35 U.S.C. § 119 to U.S. Provisional Application No. 63/685,861, filed Aug. 22, 2024, the entire disclosure of which is hereby incorporated by reference herein for all purposes.

### BACKGROUND

Vascular lesions within blood vessels and/or heart valves in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Intravascular lithotripsy is a treatment methodology that has taken on increased importance in recent years for treating vascular lesions within a body of a patient. During an intravascular lithotripsy procedure, a balloon catheter can be employed to treat the vascular lesions at a treatment site within the body of the patient. It is necessary that the balloon of the balloon catheter is maintained in a proper condition to ensure that the balloon catheter can be utilized effectively within the body of the patient.

### SUMMARY

The present invention is directed toward a protective sleeve for protecting a balloon of an intravascular lithotripsy catheter system only prior to the balloon being positioned within a body of a patient. In various embodiments, the protective sleeve includes a sleeve body that is configured to be removably positioned about the balloon. The sleeve body includes a sleeve proximal end and a sleeve distal end. The sleeve body includes a tapered region having a tapered diameter that decreases in a direction from the sleeve proximal end toward the sleeve distal end.

In some embodiments, a first diameter of the sleeve body at the sleeve proximal end is greater than a second diameter of the sleeve body at the sleeve distal end.

In certain embodiments, the tapered diameter of the tapered region decreases at a constant rate toward the sleeve distal end.

In other embodiments, the tapered diameter of the tapered region decreases at a decreasing rate toward the sleeve distal end.

In still other embodiments, the tapered diameter of the tapered region decreases at an increasing rate toward the sleeve distal end.

In some embodiments, the sleeve body further includes a proximal region that includes the sleeve proximal end.

In certain embodiments, the proximal region has a constant proximal region diameter.

In some embodiments, the tapered region of the sleeve body is adjacent to the proximal region.

In certain embodiments, the sleeve body further includes a distal region that includes the sleeve distal end.

In some embodiments, the distal region has a constant distal region diameter.

In certain embodiments, the tapered region of the sleeve body is adjacent to the distal region.

In some embodiments, the sleeve body further includes an interior region that is spaced apart from the sleeve proximal end and the sleeve distal end.

In certain embodiments, the interior region has a constant interior region diameter.

In some embodiments, the tapered region of the sleeve body is adjacent to the interior region.

In certain embodiments, the sleeve body further includes a second tapered region having a second tapered diameter that decreases toward the sleeve distal end.

In some embodiments, the second tapered diameter of the second tapered region tapers in a similar manner as the tapered diameter of the tapered region.

In other embodiments, the second tapered diameter of the second tapered region tapers in a different manner than the tapered diameter of the tapered region.

In certain embodiments, the second tapered region is spaced apart from the tapered region.

In many embodiments, the sleeve body is substantially tubular-shaped and is open at both the sleeve proximal end and the sleeve distal end.

In various embodiments, the sleeve body is formed from one or more of ultra-high molecular weight polyethylene (UHMW), high-density polyethylene (HDPE), low-density polyethylene (LDPE), nylons, polyether block amide, polyurethane, polyvinylidene fluoride, fluoropolymers, and thermoplastics.

In some embodiments, the balloon has a balloon length, and the sleeve body has a sleeve length that is at least approximately as long as the balloon length.

In certain embodiments, the balloon has a balloon length, and the sleeve body has a sleeve length that is approximately equal to the balloon length.

In some embodiments, the balloon is selectively movable between a deflated state and an inflated state, and the sleeve body being positioned about the balloon maintains the balloon in the deflated state.

In certain embodiments, the balloon has a deflated diameter when the balloon is in the deflated state, and a diameter of the sleeve body at the sleeve proximal end is at least as large as the deflated diameter.

In some embodiments, the balloon has a deflated diameter when the balloon is in the deflated state, and a diameter of the sleeve body at the sleeve proximal end is approximately equal to the deflated diameter.

The present invention is also directed toward a catheter system including a balloon that is configured to be selectively positioned within the body of the patient, and the protective sleeve as described above that is configured to be removably positioned about the balloon only prior to the balloon being positioned within the body of the patient.

In many embodiments, the balloon is selectively movable between a deflated state and an inflated state, and the balloon is configured to be positioned substantially within the sleeve body when the balloon is in the deflated state.

In certain embodiments, the catheter system further includes a catheter shaft and a guidewire lumen.

In some embodiments, the balloon is coupled to at least one of the catheter shaft and the guidewire lumen.

In other embodiments, the balloon is coupled to each of the catheter shaft and the guidewire lumen.

In certain embodiments, the balloon includes a balloon proximal end and a balloon distal end, and the balloon proximal end is coupled to the catheter shaft, and the balloon distal end is coupled to the guidewire lumen.

In some embodiments, when the balloon is positioned substantially within the sleeve body, the catheter shaft and the guidewire lumen each extend outside the sleeve body at least at one of the sleeve proximal end and the sleeve distal end.

The present invention is further directed toward a method for treating a treatment site within a body of a patient, including the steps of selectively positioning a protective sleeve about a balloon, the protective sleeve including a sleeve body having a sleeve proximal end and a sleeve distal end, the sleeve body including one or more sleeve regions including at least a tapered region having a tapered diameter that decreases toward the sleeve distal end; and removing the protective sleeve from being positioned about the balloon prior to the balloon being positioned within the body of the patient.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view illustration of an embodiment of a catheter system in accordance with various embodiments herein, the catheter system including a catheter that includes an inflatable balloon, and a protective sleeve having features of the present invention that is configured to protect the balloon prior to the catheter being utilized to treat a treatment site within a body of a patient;

FIG. 2 is a simplified schematic view illustration of a portion of an embodiment of the catheter system including an embodiment of the balloon, and an embodiment of the protective sleeve having features of the present invention;

FIG. 3 is a simplified schematic view illustration of another embodiment of the protective sleeve having features of the present invention;

FIG. 4 is a simplified schematic view illustration of still another embodiment of the protective sleeve having features of the present invention;

FIG. 5 is a simplified schematic view illustration of another embodiment of the protective sleeve having features of the present invention;

FIG. 6 is a simplified schematic view illustration of yet another embodiment of the protective sleeve having features of the present invention;

FIG. 7 is a simplified schematic view illustration of another embodiment of the protective sleeve having features of the present invention; and

FIG. 8 is a simplified schematic view illustration of still yet another embodiment of the protective sleeve having features of the present invention.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a balloon catheter configured to advance to a vascular lesion, such as a calcified vascular lesion or a fibrous vascular lesion, at a treatment site located within a body of a patient. In certain implementations, the “treatment site” can be located at or near a vessel wall of a blood vessel of the patient. Additionally, or in the alternative, in other implementations, the “treatment site” can be at or near a heart valve of the patient. Further, or in the alternative, in still other implementations, the “treatment site” can be at another suitable location within the body of the patient.

As used herein, the terms “treatment site”, “intravascular lesion” and “vascular lesion” are used interchangeably unless otherwise noted. The intravascular lesions and/or the vascular lesions are sometimes referred to herein as “lesions”.

As described in detail herein, in various embodiments, the present invention is directed toward a protective sleeve that is configured to be selectively positioned substantially about a balloon of a balloon catheter, such as can be included as part of an intravascular lithotripsy device, to provide protection for the balloon prior to the balloon catheter being utilized within the body of the patient. More particularly, the protective sleeve is configured to protect the balloon from damage, contamination, etc., which may otherwise occur in pre-use times such as during transport, storage, and the like.

It is appreciated that the balloon catheter, in general, and the protective sleeve, in particular, can be utilized as part of a treatment of the patient for peripheral artery disease (PAD) and/or coronary artery disease (CAD). It is further appreciated that by limiting passive expansion of the balloon before use through implementation and use of the protective sleeve, the balloon can possibly enter arteries that have a higher degree of calcification (clogging) since the balloon will have a smaller profile because the balloon has not been allowed to naturally expand as much prior to use in the patient.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

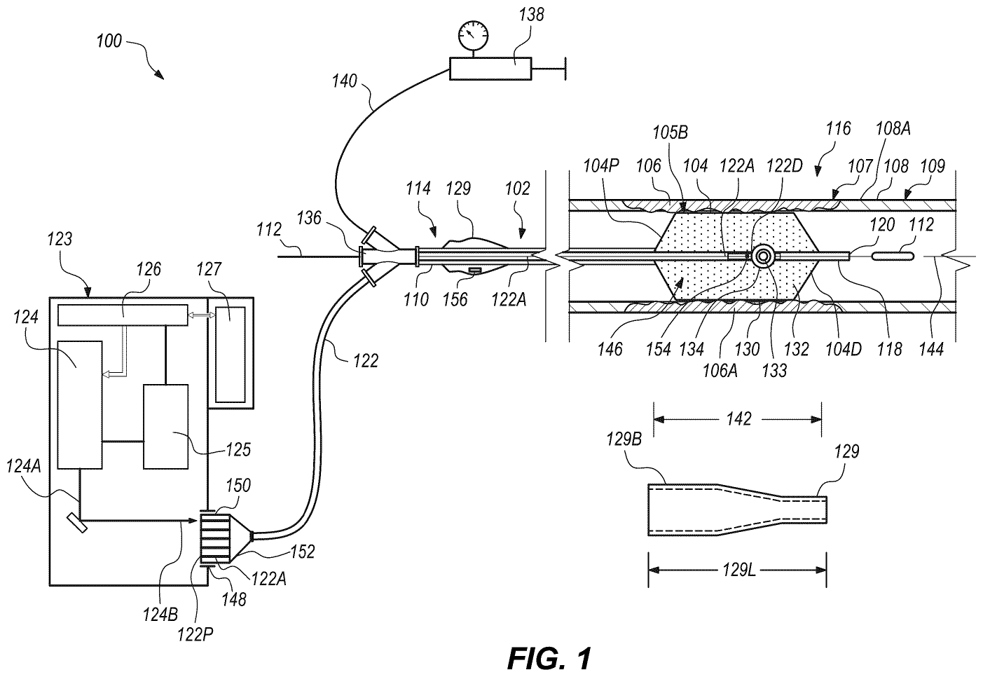

The catheter systems disclosed herein can include many different forms. Referring now to FIG. 1, a schematic cross-sectional view illustration is shown of a catheter system 100 in accordance with various embodiments. The catheter system 100 is suitable for imparting acoustic waves and/or pressure waves to induce fractures at one or more treatment sites 106 within or adjacent to a vessel wall 108A of a blood vessel 108, or on or adjacent to a heart valve, within a body 107 of a patient 109. In the embodiment illustrated in FIG. 1, the catheter system 100 can include (i) a catheter 102 including one or more of an inflatable balloon 104 (sometimes referred to herein simply as a “balloon”), a catheter shaft 110, a guidewire 112, a guidewire lumen 118, an energy guide bundle 122 including one or more energy guides 122A, a source manifold 136, a fluid pump 138, and a handle assembly 128; and (ii) a system console 123 including one or more of an energy source 124, a power source 125, a system controller 126, and a graphic user interface 127 (a “GUI”). Additionally, in various embodiments, the catheter system 100 and/or the catheter 102 further includes a protective sleeve 129 having features of the present invention that is configured to be positioned substantially about the balloon 104 to protect the balloon 104 prior to the catheter system 100 and/or the catheter 102 being used within a body 107 of a patient 109. Alternatively, the catheter system 100, the catheter 102 and/or the system console 123 can include more components or fewer components than those specifically illustrated and described in relation to FIG. 1.

As illustrated in FIG. 1, the catheter 102 is configured to move to the treatment site 106 within or adjacent to the vessel wall 108A of the blood vessel 108 within the body 107 of the patient 109. The treatment site 106 can include one or more vascular lesions 106A such as calcified vascular lesions, for example. Additionally, or in the alternative, the treatment site 106 can include vascular lesions 106A such as fibrous vascular lesions. Still alternatively, in some implementations, the catheter 102 can be used at a treatment site 106 within or adjacent to a heart valve within the body 107 of the patient 109. Yet alternatively, in certain implementations, the catheter 102 can be used at a treatment site 106 at another suitable location within the body 107 of the patient 109.

The catheter shaft 110 can extend from a proximal portion 114 of the catheter system 100 to a distal portion 116 of the catheter system 100. The catheter shaft 110 can include a longitudinal axis 144. The guidewire lumen 118 is configured to move over the guidewire 112. As utilized herein, the guidewire lumen 118 defines a conduit through which the guidewire 112 extends. The catheter shaft 110 and/or the catheter 102 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106.

The balloon 104 can include a balloon proximal end 104P and a balloon distal end 104D. In certain embodiments, the balloon 104 can be coupled to the catheter shaft 110 and/or to the guidewire lumen 118. More particularly, in some embodiments, the balloon proximal end 104P can be coupled to the catheter shaft 110, and the balloon distal end 104D can be coupled to the guidewire lumen 118.

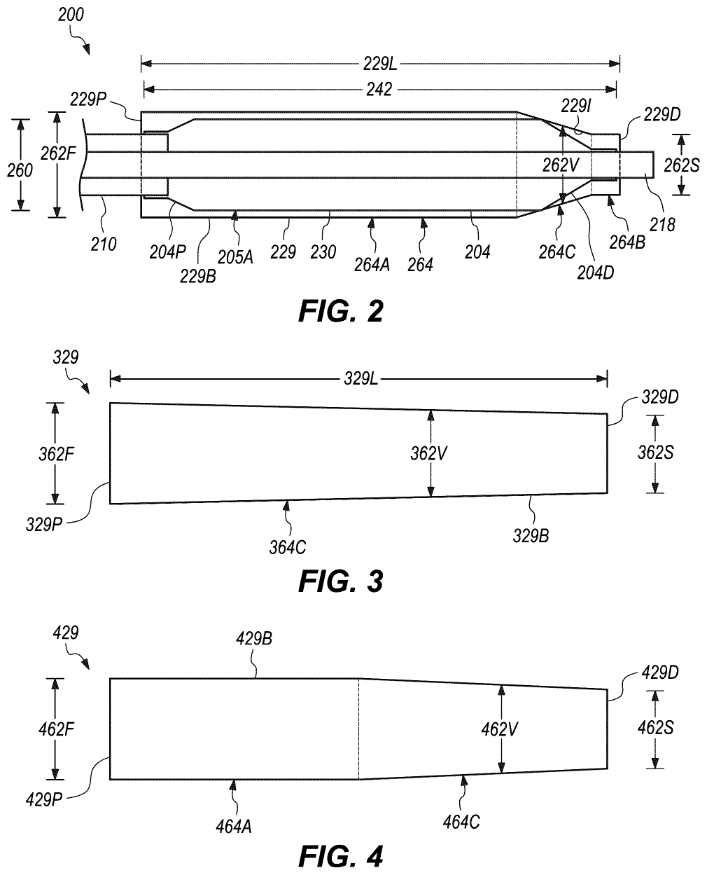

The balloon 104 includes a balloon wall 130 that defines a balloon interior 146. The balloon 104 is selectively movable between a deflated state 205A (as shown, for example, in FIG. 2) and an inflated state 105B (as shown in FIG. 1). More specifically, the balloon 104 can be selectively inflated with a catheter fluid 132 (illustrated as a plurality of small dots) to expand from the deflated state 205A, suitable for advancing the catheter 102 through a patient's vasculature, to the inflated state 105B, suitable for anchoring the catheter 102 in position relative to the treatment site 106. Stated in another manner, when the balloon 104 is in the inflated state 105B, the balloon wall 130 of the balloon 104 is configured to be positioned substantially directly adjacent to and/or in contact with the treatment site 106.

The balloon 104 suitable for use in the catheter system 100 includes those that can be passed through the vasculature of a patient 109 when in the deflated state 205A. In some embodiments, the balloon 104 is made from silicone. In other embodiments, the balloon 104 can be made from materials such as polydimethylsiloxane (PDMS), polyurethane, polymers such as a polyether block amide (such as PEBAX™) material, nylon, or any other suitable material.

The balloon 104 can have any suitable inflated diameter (in the inflated state 105B). In various embodiments, the balloon 104 can have an inflated diameter ranging from less than one millimeter (mm) up to 25 mm. In some embodiments, the balloon 104 can have an inflated diameter ranging from at least 1.5 mm up to 14 mm. In certain embodiments, the balloon 104 can have an inflated diameter ranging from at least two mm up to five mm.

The balloon 104 can also have any suitable deflated diameter (in the deflated state 205A). In many embodiments, the balloon 104 can have a deflated diameter of between approximately 0.1 mm and 1.5 mm. Alternatively, the balloon 104 can have a deflated diameter that is greater than 1.5 mm or less than 0.1 mm.

In some embodiments, the balloon 104 can have a balloon length 142 ranging from at least three mm to 300 mm. More particularly, in certain embodiments, the balloon 104 can have a balloon length 142 ranging from at least eight mm to 200 mm. As noted above, the catheter 102 and/or the balloon 104 can be utilized as part of a treatment of the patient for peripheral artery disease (PAD), which typically requires somewhat longer balloons, and/or coronary artery disease (CAD), which typically requires somewhat shorter balloons. For example, in embodiments of the balloon 104 to be used for PAD treatment, the balloon 104 can have a balloon length 142 of between approximately eight mm and 300 mm; and, in embodiments of the balloon 104 to be used for CAD treatment, the balloon 104 can have a balloon length 142 of between approximately three mm and 40 mm.

It is appreciated that a balloon 104 having a relatively longer balloon length 142 can be positioned adjacent to larger treatment sites 106, and, thus, may be usable for imparting acoustic waves and/or pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape.

In certain embodiments, the balloon 104 can have a pleated design, with a plurality of pleats that extend longitudinally along the balloon length 142 of the balloon 104, which better enables the selective inflation of the balloon 104 from the deflated state 205A to the inflated state 105B. The pleated design can further enable the balloon 104 to fit more snuggly within the protective sleeve 129 prior to use within the body 107 of the patient 109.

In some embodiments, the balloon 104 can include a drug eluting coating or a drug eluting stent structure. The drug eluting coating or drug eluting stent can include one or more therapeutic agents including anti-inflammatory agents, anti-neoplastic agents, anti-angiogenic agents, and the like.

The protective sleeve 129 can have a substantially tubular-shaped sleeve body 129B that is configured to be positioned substantially about the balloon 104 prior to the catheter 102, and thus the balloon 104, being used within the body 107 of the patient 109. Stated in another manner, prior to use within the body 107 of the patient 109, the balloon 104 can be positioned substantially within, if not entirely within, the sleeve body 129B of the protective sleeve 129. As referred to herein, the sleeve body 129B and/or the protective sleeve 129 being positioned substantially about the balloon 104 is intended to signify that the sleeve body 129B and/or the protective sleeve 129 is positioned about at least approximately 90%, 91%, 92%, 93%, 94%, 95%, 96%, 97%, 98%, or 99% of the balloon 104. As further referred to herein, the balloon 104 being positioned substantially within the sleeve body 129B and/or the protective sleeve 129 is intended to signify that at least approximately 90%, 91%, 92%, 93%, 94%, 95%, 96%, 97%, 98%, or 99% of the balloon 104 is positioned within the sleeve body 129B and/or the protective sleeve 129.

As shown in FIG. 1, the sleeve body 129B and/or the protective sleeve 129 has a sleeve length 129L that is typically at least as long, if not slightly longer, than the balloon length 142 of the balloon 104. With such design, substantially the entire balloon 104, if not completely the entire balloon 104, can be positioned within the sleeve body 129B to inhibit any potential damage, contamination, etc. from impacting the integrity of the balloon 104 prior to the balloon 104 and/or the catheter 102 being used within the body 107 of the patient 109. For example, in certain non-exclusive embodiments, the sleeve body 129B can have a sleeve length 129L of between approximately five mm and 300 mm. In embodiments of the catheter 102 to be used for PAD treatment, and where the balloon 104 is typically longer, the sleeve body 129B can have a sleeve length 129L of between approximately ten mm and 300 mm. In embodiments of the catheter 102 to be used for CAD treatment, and where the balloon 104 is typically shorter, the sleeve body 129B can have a sleeve length 129L of between approximately five mm and 40 mm. Alternatively, in some embodiments, the sleeve body 129B can have a sleeve length 129L ranging from at least ten mm to 200 mm. Still alternatively, the sleeve body 129B 129 can have another suitable sleeve length 129L, which can be greater than 300 mm or less than five mm.

It is appreciated that with the sleeve length 129L of the sleeve body 129B being approximately the same as, or only slightly longer than, the balloon length 142 of the balloon 104, the protective sleeve 129 and/or the sleeve body 129B is specifically configured to be positioned only about the balloon 104 (with only potentially very limited additional positioning about the catheter shaft 110 and/or the guidewire lumen 118) and/or to provide desired protection for only the balloon 104.

The sleeve body 129B can be made of any suitable materials. For example, in certain non-exclusive embodiments, the sleeve body 129B can be formed from one or more of ultra-high molecular weight polyethylene (UHMW), high-density polyethylene (HDPE), low-density polyethylene (LDPE), nylons, polyether block amide (such as Pebax®), polyurethane, polyvinylidene fluoride (such as Kynar®), fluoropolymers, and thermoplastics. Alternatively, the sleeve body 129B can be made from any other suitable materials.

As an overview, the present invention is directed toward a protective sleeve 129 having a substantially tubular-shaped sleeve body 129B that is open at both ends and that is tapered at some point along the sleeve length 129L of the sleeve body 129B. As shown in various embodiments illustrated and described herein below, the tapered portion of the sleeve body 129B can encompass a substantial entirety of the protective sleeve 129, or any smaller portion(s) thereof. In different embodiments, the protective sleeve 129 and/or the sleeve body 129B can also have a range of sizes (diameters) such that the sleeve body 129B is configured to fit snugly about the balloon 104 in a pre-use stage, with the balloon 104 in the deflated state 205A, so as to better protect the balloon 104 from potential damage, contamination, etc.

The catheter fluid 132 used to inflate the balloon 104 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the acoustic waves and/or pressure waves are appropriately manipulated. In certain embodiments, the catheter fluid 132 suitable for use herein is biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C₅F₁₂).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG-emission maximum=1064 nm) lasers, holmium:YAG (Ho:YAG-emission maximum=2.1 μm) lasers, or erbium:YAG (Er:YAG-emission maximum=2.94 μm) lasers. In some embodiments, the absorptive agents can be water soluble. In other embodiments, the absorptive agents are not water soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the plurality of energy guides 122A of the energy guide bundle 122 that are in optical communication with the energy source 124. The energy guide(s) 122A can be disposed along the catheter shaft 110 and within the balloon 104. Each of the energy guides 122A can have a guide distal end 122D that is at any suitable longitudinal position relative to the balloon length 142 of the balloon 104 and/or relative to a length of the guidewire lumen 118.

In some embodiments, each energy guide 122A can be an optical fiber and the energy source 124 can be a laser. The energy source 124 can be in optical communication with the energy guides 122A at the proximal portion 114 of the catheter system 100. More particularly, the energy source 124 can selectively, simultaneously, sequentially and/or alternatively be in optical communication with each of the energy guides 122A in any desired combination, sequence and/or pattern.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can be substantially annular-shaped and/or cylindrical-shaped and can have a grooved outer surface, with the grooves extending in a generally longitudinal direction along the guidewire lumen 118. In such embodiments, each of the energy guides 122A can be positioned, received and retained within an individual groove formed along and/or into the outer surface of the guidewire lumen 118. Alternatively, the guidewire lumen 118 can be formed without a grooved outer surface, and the position of the energy guides 122A relative to the guidewire lumen 118 can be maintained in another suitable manner.

The catheter system 100, the catheter 102 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100, the catheter 102 and/or the energy guide bundle 122 can include from one energy guide 122A to greater than 30 energy guides 122A. The guide distal end 122D of each of the energy guides 122A can be at any suitable or desired longitudinal position within the balloon interior 146 relative to the balloon length 142 of the balloon 104.

The energy guides 122A can have any suitable design that is useful and appropriate for purposes of enabling the generation of plasma, acoustic waves and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of enabling the generation of the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the acoustic waves and/or pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P toward the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the balloon length 142 of the balloon 104 and/or relative to the length of the guidewire lumen 118 to more effectively and more precisely impart acoustic waves and/or pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the tip of the energy guides 122A can include, but are not to be limited to, those having a convex surface, a gradient-index (GRIN) lens, and a mirror focus lens. Upon contact with the diverting structure, the energy can be diverted within the energy guide 122A to one or more of a plasma generating structure 133 (also sometimes referred to as a “plasma generator” or “plasma target”) that is positioned near, but typically spaced apart from, the guide distal end 122D of the energy guide 122A, and the photoacoustic transducer 154 that is in optical communication with a side surface of the energy guide 122A. As referred to herein, the plasma generator 133 can include and/or incorporate any suitable type of structure that is located at or near the guide distal end 122D of the energy guide 122A.

When utilized, the plasma generator 133 receives energy emitted from the guide distal end 122D of the energy guide 122A to generate plasma in the catheter fluid 132 within the balloon interior 146, which, in turn, causes the creation of plasma bubbles (cavitation bubbles) and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126 and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (which can generally include one or more ferrules) that houses a portion, such as at least the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends as part of the catheter 102 into the blood vessel 108 during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122 as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma generator 133 that can include and/or incorporate any suitable structure that is located at or near the guide distal end 122D of the energy guide 122A. In many embodiments, the plasma generator 133 can be positioned slightly spaced apart from the guide distal end 122D of the energy guide 122A. In certain embodiments, the plasma generator 133 can be provided in the form of a backstop-type structure with an angled face that redirects energy emitted from the guide distal end 122D toward the balloon wall 130 of the balloon 104 and/or toward the vessel wall 108A of the blood vessel 108 at the treatment site 106.

In particular, the energy emitted at the guide distal end 122D of the energy guide 122A is directed toward and contacts and energizes material of the plasma generator 133, such as material on an angled face of the plasma generator 133, for purposes of generating plasma in the catheter fluid 132 within the balloon interior 146. The plasma generation ionizes and superheats the surrounding catheter fluid 132 and thus causes rapid inertial bubble formation, and imparts pressure waves upon the treatment site 106. An exemplary plasma-induced bubble 134 is illustrated in FIG. 1.

The plasma generator 133 can be formed from any suitable materials. For example, in certain non-exclusive embodiments, the plasma generator 133 can be formed from one or more metals such as titanium, stainless steel, tungsten, tantalum, platinum, molybdenum, niobium, iridium, etc. Alternatively, the plasma generator 133 may be formed from plastics such as polyimide and nylon. Still alternatively, the plasma generator 133 can be formed from other suitable materials.

In various non-exclusive alternative embodiments, the sub-millisecond pulses of energy from the energy source 124 can be delivered to the treatment site 106 at a pulse frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a pulse frequency that can be greater than 5000 Hz or less than one Hz, or any other suitable range of pulse frequencies.

It is appreciated that although the energy source 124 is typically utilized to provide pulses of energy, the energy source 124 can still be described as providing a single source beam 124A, such as a single pulsed source beam.

The energy sources 124 suitable for use can include various types of light sources including lasers and lamps. For example, in certain non-exclusive embodiments, the energy source 124 can be an infrared laser that emits energy in the form of pulses of infrared light. Alternatively, as noted above, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths and energy levels that can be employed to achieve plasma and the associated cavitation bubbles in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 kHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate acoustic waves and/or pressure waves having maximum pressures in the range of at least one megapascal (MPa) to 100 MPa. The maximum pressure generated by a particular catheter system 100 will depend on the energy source 124 and the various parameters of the energy pulses from the energy source, the absorbing material, the bubble expansion, the propagation medium, the balloon material, and other factors. In various non-exclusive alternative embodiments, the catheter systems 100 can generate acoustic waves and/or pressure waves having maximum pressures in the range of at least approximately 0.1 MPa to 50 MPa, at least approximately 0.1 MPa to 30 MPa, or at least approximately 15 MPa to 25 MPa.

The acoustic waves and/or pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the acoustic waves and/or pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the acoustic waves and/or pressure waves can be imparted upon the treatment site 106 from another suitable distance that is different than the foregoing ranges. In some embodiments, the acoustic waves and/or pressure waves can be imparted upon the treatment site 106 within a range of at least approximately 0.1 MPa to 30 MPa at a distance from at least approximately 0.1 mm to ten mm. In some embodiments, the acoustic waves and/or pressure waves can be imparted upon the treatment site 106 from a range of at least approximately 0.1 MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 is electrically coupled to and receives power from the power source 125. The system controller 126 is coupled to and is configured to control operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate or frequency of pulse delivery.

The system controller 126 can further be configured to control operation of other components of the catheter system 100 such as the positioning of the catheter 102 and/or the guide distal end 122D of the energy guides 122A adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is attached to the catheter shaft 110 and is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127.

In some embodiments, the handle assembly 128 can integrate and/or include at least a portion of the system controller 126 within an interior of the handle assembly 128. For example, as shown, in certain embodiments, the handle assembly 128 can include circuitry 156, which is electrically coupled between catheter electronics and the system console 123, and which can form at least a portion of the system controller 126. In some embodiments, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

As with all embodiments illustrated and described herein, various structures may be omitted from the figures for clarity and ease of understanding. Further, the figures may include certain structures that can be omitted without deviating from the intent and scope of the invention.

FIG. 2 is a simplified schematic view illustration of a portion of an embodiment of the catheter system 200 including an embodiment of the balloon 204, and an embodiment of the protective sleeve 229 having features of the present invention. More particularly, FIG. 2 shows a portion of a catheter shaft 210 and a portion of a guidewire lumen 218 of the catheter system 200, as well as the balloon 204 and the protective sleeve 229.

As shown in FIG. 2, in many embodiments, the balloon 204 has a balloon proximal end 204P that is coupled and/or secured to the catheter shaft 210, and a balloon distal end 204D that is coupled and/or secured to the guidewire lumen 218.

In FIG. 2, the balloon 204 is shown in the deflated state 205A, as that is the state of the balloon 204 when the balloon 204 is positioned within the sleeve body 229B of the protective sleeve 229. In some embodiments, as noted above, the balloon 204 can have a pleated design, with a plurality of pleats extending longitudinally along the balloon length 242, which better enables movement between the deflated state 205A and the inflated state 105B. As further noted above, the pleated design also better enables the balloon 204 to fit more snuggly within the sleeve body 229B prior to the balloon 204 being used within the body 107 (illustrated in FIG. 1) of the patient 109 (illustrated in FIG. 1).

The balloon 204 can have any suitable deflated (outer) diameter 260 (in the deflated state 205A). In many embodiments, the balloon 204 can have a deflated diameter 260 of between approximately 0.1 mm and 1.5 mm. Alternatively, the balloon 204 can have a deflated diameter 260 that is greater than 1.5 mm or less than 0.1 mm.

It is appreciated that in order for the balloon 204 in the deflated state 205A to fit snugly within the sleeve body 229B, the sleeve body 229B would have a sleeve inner diameter 262 that is approximately equal to, or only slightly larger than, the deflated diameter 260 of the balloon 204. For example, in many embodiments, the sleeve body 229B can have a sleeve inner diameter 262 that is also between approximately 0.1 mm and 1.5 mm. Alternatively, the sleeve body 229B can have a sleeve inner diameter 262 that is greater than 1.5 mm or less than 0.1 mm.

As referred to herein, in many implementations, the sleeve inner diameter 262 can be equated with the maximum inner diameter of the sleeve body 229B at any point along a sleeve length 229L of the sleeve body 229B. Based on the design of the protective sleeve 229, in various embodiments, the sleeve inner diameter 262 will often be equated with a first diameter 262F of the sleeve body 229B, which is the inner diameter of the sleeve body 229B at a sleeve proximal end 229P.

The balloon 204 can have any suitable ballon length 242 for purposes of treating the treatment site 106 (illustrated in FIG. 1) and/or the vascular lesions 106A (illustrated in FIG. 1) within the body 107 of the patient 109. The balloon 204 will typically be configured such that the balloon length 242 is approximately the same regardless of whether the balloon 204 is in the deflated state 205A (as shown in FIG. 2) or the inflated state 105B (as shown in FIG. 1). Stated in another manner, in most embodiments, the balloon length 242 will not change as the balloon 204 is moved between the deflated state 205A and the inflated state 105B.

As with the embodiments illustrated and described herein above, in certain embodiments, the balloon 204 can have a balloon length 242 ranging from at least three mm to 300 mm. As noted above, the catheter system 200 and/or the balloon 204 can be utilized as part of a treatment of the patient for peripheral artery disease (PAD), which typically requires somewhat longer balloons, and/or coronary artery disease (CAD), which typically requires somewhat shorter balloons. For example, in embodiments of the balloon 204 to be used for PAD treatment, the balloon 204 can have a balloon length 242 of between approximately eight mm and 300 mm; and, in embodiments of the balloon 204 to be used for CAD treatment, the balloon 204 can have a balloon length 242 of between approximately three mm and 40 mm.

Alternatively, the balloon 204 can have another suitable balloon length 242 that can be greater than approximately 300 mm or less than approximately three mm.

The protective sleeve 229 is configured to provide desired and necessary protection for the balloon 204 while the balloon 204 is in the deflated state 205A and before the balloon 204 is utilized as part of the catheter system 200 within the body 107 of the patient 109. In various embodiments, the protective sleeve 229 can have a substantially tubular-shaped sleeve body 229B, having an open sleeve proximal end 229P and an opposed, open sleeve distal end 229D. The sleeve body 229B can be positioned substantially about the balloon 204 (with only potentially very limited additional positioning about the catheter shaft 210 and/or the guidewire lumen 218), with the balloon 204 positioned substantially therewithin. With such design, the protective sleeve 229 is generally configured to provide desired and necessary protection for only the balloon 204.

As shown in FIG. 2, the sleeve body 229B has a sleeve length 229L that is approximately equal to, if not slightly longer than, the balloon length 242 of the balloon 204. Stated in another manner, with the balloon 204 being positioned substantially, if not entirely, within the sleeve body 229 of the protective sleeve 229, the sleeve body 229B can have the sleeve proximal end 229P that substantially coincides with the balloon proximal end 204P, and the sleeve distal end 229D that substantially coincides with the balloon distal end 204D.

In some non-exclusive embodiments, when the balloon 204 is positioned substantially within the sleeve body 229B, the sleeve distal end 229D can extend longitudinally between approximately 0.1 mm and one millimeter beyond the balloon distal end 204D. Alternatively, the sleeve distal end 229D can extend greater than one millimeter or less than 0.1 mm beyond the balloon distal end 204D.

Somewhat similarly, in certain non-exclusive embodiments, when the balloon 204 is positioned substantially within the sleeve body 229B, the sleeve proximal end 229P can extend longitudinally between approximately 0.1 mm and one millimeter beyond the balloon proximal end 204P. Alternatively, the sleeve proximal end 229P can extend greater than one millimeter or less than 0.1 mm beyond the balloon proximal end 204P.

As with the embodiments illustrated and described herein above, in certain embodiments wherein the sleeve length 229L of the sleeve body 229B is approximately equal to or slightly longer than the balloon length 242 of the balloon 204, the sleeve body 229B can have a sleeve length 229L of between approximately five mm and 300 mm. More particularly, in embodiments where the catheter system 200 is to be used for PAD treatment, the sleeve body 229B can have a sleeve length 229L of between approximately ten mm and 300 mm; and, in embodiments where the catheter system 200 is to be used for CAD treatment, the sleeve body 229B can have a sleeve length 229L of between approximately five mm and 40 mm. Alternatively, the sleeve body 229B can have another suitable sleeve length 229L that can be greater than approximately 300 mm or less than approximately five mm.

It is further recognized that with the protective sleeve 229 being configured specifically to provide desired and necessary protection for only the balloon 204, portions of the catheter shaft 210 and the guidewire lumen 218 will extend beyond the confines of the sleeve body 229B. Stated in another manner, as illustrated, when the balloon 204 is positioned substantially within the sleeve body 229B, portions of the catheter shaft 210 and the guidewire lumen 218 extend longitudinally outside the sleeve body 229B at least at one, if not both in the case of the guidewire lumen 218, of the sleeve proximal end 229P and the sleeve distal end 229D.

The protective sleeve 229 can have any suitable design for purposes of providing the desired and necessary protection of the balloon 204 from damage, contamination, etc. during transport, storage, and the like, prior to the balloon 204 being used within the body 107 of the patient 109. More particularly, the protective sleeve 229 can have any suitable design that enables the balloon 204, in the deflated state 205A, to fit snugly therewithin, while also enabling the balloon 204 to be quickly and easily removed from within the protective sleeve 229.

In various embodiments, the protective sleeve 229 and/or the sleeve body 229B includes one or more sleeve regions 264 that are defined along and/or relative to the sleeve length 229L of the sleeve body 229B, including at least one tapered sleeve region that gets smaller or decreases in diameter when moving closer to the sleeve distal end 229D. For example, in the embodiment illustrated in FIG. 2, the protective sleeve 229 and/or the sleeve body 229B includes a first (proximal) sleeve region 264A (also sometimes referred to as a “first region” or a “proximal region”) that includes the sleeve proximal end 229P, a second (distal) sleeve region 264B (also sometimes referred to as a “second region” or a “distal region”) that includes the sleeve distal end 229D, and a third (tapered) sleeve region 264C (also referred to generally as a “tapered region”) that extends from and between the first region 264A and the second region 264B. As shown, the first (proximal) sleeve region 264A has a constant, first diameter 262F (that is equivalent to the sleeve inner diameter 262), and the second (distal) sleeve region 264B has a constant, second diameter 262S that is less than the first diameter 262F. The tapered region 264C has a decreasing, tapered diameter 262V moving from the connection with the first sleeve region 264A to the connection with the second sleeve region 264B. As such, the tapered diameter 262V of the tapered region 264C gradually decreases toward the sleeve distal end 229D, from being equal to the first diameter 262F to being equal to the second diameter 262S as the tapered region 264C extends from the first (proximal) sleeve region 264A to the second (distal) sleeve region 264B. In the embodiment shown in FIG. 2, the tapered diameter 262V of the tapered region 264C decreases at a constant rate from the first diameter 262F (adjacent to the first region 264A) to the second diameter 262S (adjacent to the second region 264B). Alternatively, the tapered diameter 262V of the tapered region 264C can decreasing at a non-constant rate, either an increasing rate of change or a decreasing rate of change, from the first diameter 262F (adjacent to the first region 264A) to the second diameter 262S (adjacent to the second region 264B).

It is appreciated that the lengths of each of the sleeve regions 264A-264C can be varied to suit the requirements of the balloon 204, individually, and/or the catheter system 200, as a whole. The delineation between adjacent sleeve regions 264 is shown in FIG. 2 with a pair of dotted lines. In alternative embodiments, the sleeve body 229B can have more than three sleeve regions 264 or fewer than three sleeve regions 264, as long as there is at least one tapered region. For example, in one non-exclusive alternative embodiment, the sleeve body 229B can include just a single tapered region that extends the entire sleeve length of the protective sleeve 229. In other non-exclusive alternative embodiments, the sleeve body 229B can include more than one tapered region.

During use of the protective sleeve 229, the balloon 204 is inserted into the sleeve body 229B, and/or the sleeve body 229B is moved to be positioned about the balloon 204, with the balloon distal end 204D initially entering into the sleeve body 229B through the sleeve proximal end 229P. The relative movement between the balloon 204 and the sleeve body 229B continues until a portion of the balloon wall 230 engages and/or contacts an inner surface 2291 of the sleeve body 229B such that the balloon 204 can move no further within the sleeve body 229B. In the embodiment shown in FIG. 2, the balloon 204 continues its movement relative to the sleeve body 229B until a portion of the balloon wall 230 engages and/or contacts the inner surface 2291 of the sleeve body 229B within the tapered region 264C. As shown, at the extent of this relative movement between the balloon 204 and the sleeve body 229B, the balloon distal end 204D is inhibited from extending beyond the sleeve distal end 229D. Moreover, with the balloon 204 now positioned at least substantially within the sleeve body 229B, the balloon distal end 204D substantially coincides with the sleeve distal end 229D, and the balloon proximal end 204P substantially coincides with the sleeve proximal end 229P. As utilized herein, the balloon distal end 204D substantially coinciding with the sleeve distal end 229D, and the balloon proximal end 204P substantially coinciding with the sleeve proximal end 229P, means that the corresponding ends are longitudinally within less than approximately 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, or 2.0 mm of one another.

It is appreciated that, with this design, when it is time to utilize the catheter system 200 for an intravascular lithotripsy procedure, the balloon 204 can be quickly and easily removed from the protective sleeve 229 and/or the sleeve body 229B through the sleeve proximal end 229P. More specifically, as shown, since the first diameter of the first region 264A of the sleeve body 229B is at least as large as, if not slightly larger than, the deflated diameter of the balloon 224, the balloon 204 can be quickly and easily removed from the sleeve body 229B through the sleeve proximal end 229P while only experiencing a limited amount of frictional resistance.

The protective sleeve 229 and/or the sleeve body 229B can be made of any suitable materials. For example, in certain non-exclusive embodiments, the protective sleeve 229 and/or the sleeve body 229B can be formed from one or more of ultra-high molecular weight polyethylene (UHMW), high-density polyethylene (HDPE), low-density polyethylene (LDPE), nylons, polyether block amide (such as Pebax®), polyurethane, polyvinylidene fluoride (such as Kynar®), fluoropolymers, and thermoplastics. Alternatively, the protective sleeve 229 and/or the sleeve body 229B can be made from any other suitable materials.

FIG. 3 is a simplified schematic view illustration of another embodiment of the protective sleeve 329 having features of the present invention. In this embodiment, the protective sleeve 329 is again configured to provide desired and necessary protection for the balloon 204 (illustrated in FIG. 2) prior to the balloon 204 being used within the body 107 (illustrated in FIG. 1) of the patient 109 (illustrated in FIG. 1). Stated in another manner, when the balloon 204 is in the deflated state 205A (illustrated in FIG. 2), the protective sleeve 329 is again configured such that the balloon 204 fits substantially, if not entirely, within the sleeve body 329B of the protective sleeve 329.

However, as shown in FIG. 3, the protective sleeve 329 has a different design than in the previously described embodiments. In particular, in the embodiment illustrated in FIG. 3, the sleeve body 329B again includes a sleeve proximal end 329P and a sleeve distal end 329D, but only includes a single tapered region 364C that extends the full sleeve length 329L of the sleeve body 329B from the sleeve proximal end 329P to the sleeve distal end 329D. As shown, in one embodiment, the tapered region 364C can have a tapered diameter 362V that gradually decreases at a constant rate from a first diameter 362F, at the sleeve proximal end 329P, to a second diameter 362S, at the sleeve distal end 329D. Alternatively, the tapered region 364C can have a tapered diameter 362V that decreases at a non-constant rate from the first diameter 362F to the second diameter 362S.

FIG. 4 is a simplified schematic view illustration of still another embodiment of the protective sleeve 429 having features of the present invention. In this embodiment, the protective sleeve 429 is again configured to provide desired and necessary protection for the balloon 204 (illustrated in FIG. 2) prior to the balloon 204 being used within the body 107 (illustrated in FIG. 1) of the patient 109 (illustrated in FIG. 1). Stated in another manner, when the balloon 204 is in the deflated state 205A (illustrated in FIG. 2), the protective sleeve 429 is again configured such that the balloon 204 fits substantially, if not entirely, within the sleeve body 429B of the protective sleeve 429.

As shown in FIG. 4, the protective sleeve 429 has a different design than in the previously described embodiments. In particular, in the embodiment illustrated in FIG. 4, the sleeve body 429B again includes a sleeve proximal end 429P and a sleeve distal end 429D. However, in this embodiment, the sleeve body 429B includes a first (proximal) region 464A that includes the sleeve proximal end 429P, the first region 464A including a constant, first diameter 462F, and a tapered region 464C that extends from the first region 464A to the sleeve distal end 429D. The delineation between the first region 464A and the tapered region 464C is shown in FIG. 4 with a dotted line.

As shown, in one embodiment, the tapered region 464C can have a tapered diameter 462V that gradually decreases at a constant rate from the first diameter 462F, at the sleeve proximal end 429P and throughout the first region 464A, to a second diameter 462S, at the sleeve distal end 429D. Alternatively, the tapered region 464C can have a tapered diameter 462V that decreases at a non-constant rate from the first diameter 462F to the second diameter 462S.

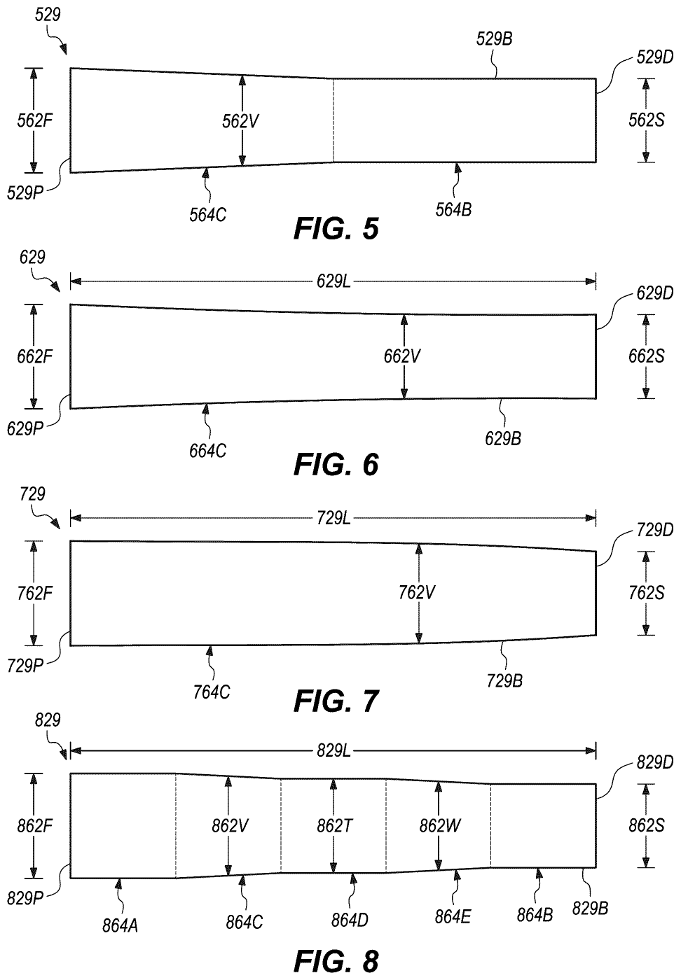

FIG. 5 is a simplified schematic view illustration of another embodiment of the protective sleeve 529 having features of the present invention. In this embodiment, the protective sleeve 529 is again configured to provide desired and necessary protection for the balloon 204 (illustrated in FIG. 2) prior to the balloon 204 being used within the body 107 (illustrated in FIG. 1) of the patient 109 (illustrated in FIG. 1). Stated in another manner, when the balloon 204 is in the deflated state 205A (illustrated in FIG. 2), the protective sleeve 529 is again configured such that the balloon 204 fits substantially, if not entirely, within the sleeve body 529B of the protective sleeve 529.

As shown in FIG. 5, the protective sleeve 529 has a different design than in the previously described embodiments. In particular, in the embodiment illustrated in FIG. 5, the sleeve body 529B again includes a sleeve proximal end 529P and a sleeve distal end 529D. However, in this embodiment, the sleeve body 529B includes a tapered region 564C that includes the sleeve proximal end 529P, and a second (distal) region 564B that includes the sleeve distal end 529D, and that extends from the tapered region 564C to the sleeve distal end 529D, the second region 564B including a constant, second diameter 562S. The delineation between the tapered region 564C and the second region 564B is shown in FIG. 5 with a dotted line.

As shown, in one embodiment, the tapered region 564C can have a tapered diameter 562V that gradually decreases at a constant rate from a first diameter 562F, at the sleeve proximal end 529P, to the second diameter 562S, at the sleeve distal end 529D and throughout the second region 564B. Alternatively, the tapered region 564C can have a tapered diameter 562V that decreases at a non-constant rate from the first diameter 562F to the second diameter 562S.

FIG. 6 is a simplified schematic view illustration of yet another embodiment of the protective sleeve 629 having features of the present invention. In this embodiment, the protective sleeve 629 is again configured to provide desired and necessary protection for the balloon 204 (illustrated in FIG. 2) prior to the balloon 204 being used within the body 107 (illustrated in FIG. 1) of the patient 109 (illustrated in FIG. 1). Stated in another manner, when the balloon 204 is in the deflated state 205A (illustrated in FIG. 2), the protective sleeve 629 is again configured such that the balloon 204 fits substantially, if not entirely, within the sleeve body 629B of the protective sleeve 629.

As shown in FIG. 6, the protective sleeve 629 has a different design than in the previously described embodiments. In particular, in the embodiment illustrated in FIG. 6, the sleeve body 629B again includes a sleeve proximal end 629P and a sleeve distal end 629D. In this embodiment, similar to the embodiment illustrated and described in relation to FIG. 3, the sleeve body 629B again includes a single tapered region 664C that extends the full sleeve length 629L of the sleeve body 629B from the sleeve proximal end 629P to the sleeve distal end 629D. However, in this embodiment, the tapered region 664C has a tapered diameter 662V that decreases at a non-constant, decreasing rate from a first diameter 662F, at the sleeve proximal end 629P, to a second diameter 662S, at the sleeve distal end 629D.

FIG. 7 is a simplified schematic view illustration of another embodiment of the protective sleeve 729 having features of the present invention. In this embodiment, the protective sleeve 729 is again configured to provide desired and necessary protection for the balloon 204 (illustrated in FIG. 2) prior to the balloon 204 being used within the body 107 (illustrated in FIG. 1) of the patient 109 (illustrated in FIG. 1). Stated in another manner, when the balloon 204 is in the deflated state 205A (illustrated in FIG. 2), the protective sleeve 729 is again configured such that the balloon 204 fits substantially, if not entirely, within the sleeve body 729B of the protective sleeve 729.

As shown in FIG. 7, the protective sleeve 729 has a different design than in the previously described embodiments. In particular, in the embodiment illustrated in FIG. 7, the sleeve body 729B again includes a sleeve proximal end 729P and a sleeve distal end 729D. In this embodiment, similar to the embodiment illustrated and described in relation to FIG. 3, the sleeve body 729B again includes a single tapered region 764C that extends the full sleeve length 729L of the sleeve body 729B from the sleeve proximal end 729P to the sleeve distal end 729D. However, in this embodiment, the tapered region 764C has a tapered diameter 762V that decreases at a non-constant, increasing rate from a first diameter 762F, at the sleeve proximal end 729P, to a second diameter 762S, at the sleeve distal end 729D.

FIG. 8 is a simplified schematic view illustration of still yet another embodiment of the protective sleeve 829 having features of the present invention. In this embodiment, the protective sleeve 829 is again configured to provide desired and necessary protection for the balloon 204 (illustrated in FIG. 2) prior to the balloon 204 being used within the body 107 (illustrated in FIG. 1) of the patient 109 (illustrated in FIG. 1). Stated in another manner, when the balloon 204 is in the deflated state 205A (illustrated in FIG. 2), the protective sleeve 829 is again configured such that the balloon 204 fits substantially, if not entirely, within the sleeve body 829B of the protective sleeve 829.

As shown in FIG. 8, the protective sleeve 829 has a different design than in the previously described embodiments. In particular, in the embodiment illustrated in FIG. 8, the sleeve body 829B again includes a sleeve proximal end 829P and a sleeve distal end 829D. Similar to the embodiment shown in FIG. 2, in this embodiment, the sleeve body 829B includes a first (proximal) region 864A that includes the sleeve proximal end 829P and has a constant first diameter 862F, and a second (distal) region 864B that includes the sleeve distal end 829D and has a constant second diameter 862S that is different than (less than) the first diameter 862F.

However, in this embodiment, rather than including a single tapered region as in FIG. 2, the sleeve body 829B further includes a first tapered region 864C, a third (interior) region 864D that has a constant third diameter 862T that is different than (in between) the first diameter 862F and the second diameter 862S, and a second tapered region 864E. As shown, the first tapered region 864C extends from and between the first (proximal) region 864A and the third (interior) region 864D; and the second tapered region 864E extends from and between the third (interior) region 864D and the second (distal) region 864B. The delineation between the regions 864A-864E is shown in FIG. 8 with a series of dotted lines.

It is appreciated that in the embodiment illustrated in FIG. 8, the first tapered region 864C and the second tapered region 864E are spaced apart from one another along the sleeve length 829L of the sleeve body 829B. However, in alternative embodiments, the first tapered region 864C and the second tapered region 864E can be adjacent to one another along the sleeve length 829L of the sleeve body 829B.

As shown, in one embodiment, the first tapered region 864C can have a first tapered diameter 862V that gradually decreases at a constant rate from the first diameter 862F to the third diameter 862T. Alternatively, the first tapered region 864C can have a first tapered diameter 862V that decreases at a non-constant (increasing or decreasing) rate from the first diameter 862F to the third diameter 862T.

Similarly, as shown, in one embodiment, the second tapered region 864E can have a second tapered diameter 862W that gradually decreases at a constant rate from the third diameter 862T to the second diameter 862S. Alternatively, the second tapered region 864E can have a second tapered diameter 862W that decreases at a non-constant (increasing or decreasing) rate from the third diameter 862T to the second diameter 862S.

It is appreciated that, in alternative embodiments, the first tapered diameter 862V of the first tapered region 864C can taper in a similar manner (at substantially the same rate) as the second tapered diameter 862W of the second tapered region 864E; or the first tapered diameter 862V of the first tapered region 864C can taper in a different manner (at a different rate) than the second tapered diameter 862W of the second tapered region 864E.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system, the catheter, and/or the protective sleeve have been illustrated and described herein, one or more features of any one embodiment can be combined with one or more features of one or more of the other embodiments, provided that such combination satisfies the intent of the present invention.

While a number of exemplary aspects and embodiments of the catheter system, the catheter, and/or the protective sleeve have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A protective sleeve for protecting a balloon of an intravascular lithotripsy catheter system only to the balloon being positioned within a body of a patient, the protective sleeve comprising:
a sleeve body that is configured to be removably positioned about the balloon, the sleeve body including a sleeve proximal end and a sleeve distal end, the sleeve body including a tapered region having a tapered diameter that decreases in a direction from the sleeve proximal end toward the sleeve distal end.

2. The protective sleeve of claim 1, wherein the tapered diameter of the tapered region decreases at a constant rate toward the sleeve distal end.

3. The protective sleeve of claim 1, wherein the tapered diameter of the tapered region decreases at a decreasing rate toward the sleeve distal end.

4. The protective sleeve of claim 1, wherein the tapered diameter of the tapered region decreases at an increasing rate toward the sleeve distal end.

5. The protective sleeve of claim 1, wherein the sleeve body further includes a proximal region that includes the sleeve proximal end and the proximal region has a constant proximal region diameter.

6. The protective sleeve of claim 1, wherein the sleeve body further includes a distal region that includes the sleeve distal end and the distal region has a constant distal region diameter.

7. The protective sleeve of claim 1, wherein the sleeve body further includes an interior region that is spaced apart from the sleeve proximal end and the sleeve distal end and the interior region has a constant interior region diameter.

8. The protective sleeve of claim 7, wherein the tapered region of the sleeve body is adjacent to the interior region.

9. The protective sleeve of claim 1, wherein the sleeve body further includes a second tapered region having a second tapered diameter that decreases toward the sleeve distal end.

10. A catheter system comprising:
a balloon that is configured to be selectively positioned within a body of a patient; and
a protective sleeve that is configured to be removably positioned about the balloon prior to the balloon being positioned within the body of the patient, wherein the protective sleeve comprises:
a sleeve body, the sleeve body including a sleeve proximal end, a sleeve distal end, the sleeve body, and a tapered region having a tapered diameter that decreases in a direction from the sleeve proximal end toward the sleeve distal end.

11. The catheter system of claim 10, wherein the balloon is selectively movable between a deflated state and an inflated state, and wherein the balloon is configured to be positioned substantially within the sleeve body when the balloon is in the deflated state.

12. The catheter system of claim 10, further comprising:
a catheter shaft, and
wherein the balloon is coupled to the catheter shaft.

13. The catheter system of claim 12, wherein the balloon includes a balloon proximal end and a balloon distal end, and wherein the balloon proximal end is coupled to the catheter shaft.

14. The catheter system of claim 12, wherein when the balloon is positioned substantially within the sleeve body, the catheter shaft extends outside of the sleeve body at least at one of the sleeve proximal end and the sleeve distal end.

15. A method for treating a treatment site within a body of a patient, the method comprising:
selectively positioning a protective sleeve about a balloon, the protective sleeve including a sleeve body having a sleeve proximal end and a sleeve distal end, the sleeve body including one or more sleeve regions including at least a tapered region having a tapered diameter that decreases toward the sleeve distal end; and
removing the protective sleeve from being positioned about the balloon prior to the balloon being positioned within the body of the patient.

16. The method of claim 15, wherein selectively positioning includes a first diameter of the sleeve body at the sleeve proximal end being greater than a second diameter of the sleeve body at the sleeve distal end.

17. The method of claim 15, wherein selectively positioning includes the tapered diameter of the tapered region decreasing at a constant rate toward the sleeve distal end.

18. The method of claim 15, wherein selectively positioning includes the tapered diameter of the tapered region decreasing at a decreasing rate toward the sleeve distal end.

19. The method of claim 15, wherein selectively positioning includes the sleeve body further including a proximal region that includes the sleeve proximal end and the proximal region having a constant proximal region diameter.

20. The method of claim 15, wherein selectively positioning includes the sleeve body further including a distal region that includes the sleeve distal end and the distal region having a constant distal region diameter.
